(19) United States
(12) Patent Application Publication
(10) Pub. No.: US 2010/0082097 A1
(43) Pub. Date: Apr. 1, 2010

(54) Article Containing Segregated Biguanide and Lewis Acid

(76) Inventors: Joel Rosenblatt, Pottstown, PA (US); Perry G. Wang, Lake Villa, IL (US); Hiep Do, Sinking Spring, PA (US)

Correspondence Address: BAKER & HOSTETLER LLP, WASHINGTON SQUARE, SUITE 1100, 1050 CONNECTICUT AVE. N.W., WASHINGTON, DC, 20036-5304, US

(21) Appl. No.: 12/570,707

(22) Filed: Sep. 30, 2009

Related U.S. Application Data
(60) Provisional application No. 61/101,903, filed on Oct. 1, 2008.

(51) Int. Cl. A61L 27/50; A61L 29/14; A61L 17/00; A61L 31/14; B29C 47/06

(52) U.S. Cl. 623/1.46; 604/265; 606/231; 606/151; 604/8; 264/173.16

(57) ABSTRACT

A medical device includes a first region having a biguanide or a pharmaceutically acceptable salt thereof and a second region having a Lewis acid.

### CROSS-REFERENCE TO RELATED APPLICATIONS

This application claims priority to provisional U.S. patent application filed Oct. 1, 2008, having a Ser. No. 61/101,903, the disclosure of which is hereby incorporated by reference in its entirety.

### FIELD OF THE INVENTION

The present invention generally relates to medical devices having antimicrobial properties. More particularly, the present invention pertains to medical articles having a plurality of antimicrobial agents combined therein in a novel manner.

### BACKGROUND OF THE INVENTION

In various medical procedures, medical devices having antimicrobial properties are used to reduce the incidence of infection in the patient. Examples of such devices include catheters, grafts, stents, sutures, and the like. In conventional medical articles having antimicrobial properties, the antimicrobial agent is generally a broad spectrum agent. To further reduce incidence of infection, two or more different antimicrobial agents have been proposed for use with medical articles. It has been found that a particularly beneficial combination of antimicrobial agents includes chlorhexidine and Gentian violet.

The benefits of chlorhexidine salts as antiseptics used to combat infection are widely established. See: Maki, D. G., M. Ringer, and C. J. Alvarado. 1991. Prospective randomized trial of povidone-iodine, alcohol, and chlorhexidine for prevention of infection associated with central venous and arterial catheters. Lancet 338:339-343; Greenfield, J. I., Sampath, L., Popilskis, S. J. et al. 1995. Decreased bacterial adherence and biofilm formation on chlorhexidine and silver sulfadiazine impregnated central venous catheters implanted in swine. Critical Care Medicine 23, 894-900; Veenstra, D. O., Saint, S., Saha, S. et al. 1999. Efficacy of antiseptic-impregnated ventral venous catheters in preventing catheter-related bloodstream infection: a meta analysis. Journal of the American Medical Association 281, 261-267; Gaonkar, T. A. and Modak, S. M. 2003. Comparison of microbial adherence to antiseptic and antibiotic central venous catheters using a novel agar subcutaneous infection model. Journal of Antimicrobial Chemotherapy 52, 389-396; Loe, H. 1973. Chlorhexidine in prophylaxis of dental diseases. Introduction. J Periodontal Res. 8(Supp 12), 5-6 and; Crosfill, M., Hall, R., London, D. 1969. The use of chlorhexidine antisepsis in contaminated surgical wounds. Br J. Surg. 56, 906-908. Briefly, chlorhexidine is a biguanide with broad spectrum efficacy against both gram negative and gram positive bacteria. See: Bassetti, S., Hu, J., D'Agostino, R. B., Jr et al. 2001. Prolonged antimicrobial activity of a catheter containing chlorhexidine-silver sulfadiazine extends protection against catheter infections in vivo. Antimicrobial Agents and Chemotherapy 45, 1535-1538; Maki, D. G., Stoltz, S. M., Wheeler, S. et al. 1997. Prevention of central venous catheter-related bloodstream infection by use of an antiseptic-impregnated catheter: a randomized controlled trial. Annals of Internal Medicine 127, 257-266 and; Gardner, J. F. and K. G. Gray. Chlorhexidine. In: Block Ss, ed. Disinfection, Sterilization and Preservation. Philadelphia: Lea and Febiger; 1983:251-270. Chlorhexidine has proven to be clinically effective as a treatment on a catheter surface in reducing catheter related bloodstream infections (CR-BSIs), saving thousands of dollars a year in cost of infection treatment in hospitals. See: Hanna, H., Bahna, P., Reitzel, R., Dvorak, T, Chaiban, G., Hachem, R., Raad, I. 2006. Comparative in vitro efficacies and antimicrobial durabilities of novel antimicrobial central venous catheters. Antimicrobial Agents and Chemotherapy 50, 3283-3288; and Rupp, M. E., S. J. Lisco, P. A. Lipsett, T. M. Pearl, K. Keating, J. M. Civetta, L. A. Mermel, D. Lee, E. P. Dellinger, M. Donahoe, D. Giles, M. A. Pfaller, D. G. Maki, and R. Sherertz. 2005. Effect of a second generation venous catheter impregnated with chlorhexidine and silver sulfadiazine on central catheter-related infections: a randomized, controlled trial. Annals of Internal Medicine, 143, 570-580.

Gentian violet (GV) is a triphenylmethane dye that can be used as an antiseptic against fungi and gram positive bacteria such as Staphylococcus aureus. See: Saji, M., Taguchi, S., Uchiyama, K., Osono, E., Hayama, N., and H. Ohkuni. 1995. Efficacy of Gentian violet in the eradication of methicillin-resistant Staphylococcus aureus from skin lesions. Journal of Hospital Infection, 31, 225-228. Historically, GV is associated with its use in prevention of infections caused by Candida albicans and other species of Candida. See: Sutton, R. L. 1938. Gentian violet as a therapeutic agent with note on a case of Gentian violet tattoo. Journal of the American Medical Association 100, 1733-1738. Candida albicans accounts for about 8% of hospital acquired bloodstream infections per year, and has a mortality rate of 40%. See: Edmond M B, Wallace S E, McClish D K, et al. Nosocomial bloodstream infections in United States hospitals: a three-year analysis. Clin Infect Dis 1999; 29:239-44.

Combinations of chlorhexidine and GV have broad spectrum antimicrobial efficacy against gram negative and gram positive bacteria, as well as yeasts. See: Hanna, H., Bahna, P., Reitzel, R., Dvorak, T, Chaiban, G., Hachem, R., Raad, I. 2006. Comparative in vitro efficacies and antimicrobial durabilities of novel antimicrobial central venous catheters. Antimicrobial Agents and Chemotherapy 50, 3283-3288. The combination as a catheter coating has also shown to be more effective against methicillin-resistant Staphylococcus aureus (MRSA), Pseudomonas aeruginosa, and Candida parapsilosis than the catheters coated with chlorhexidine and silver sulfadiazine, or minocycline and rifampin. See: Raad, et al. U.S. Patent Application Publication US 2003/0078242 A1.

Unfortunately, some combinations of antimicrobial agents interact negatively. These negative interactions may include inactivation of one or both agents, decreases in the stability of one or both agents and the like. More particularly, degradation of one or both agents may present safety issues such as, for example, irritation, allergenicity, and/or toxicity.

In the case of chlorhexidine and GV, freely mixing the two agents produces an undesirable chlorhexidine degradation product. Production of this degradation product is enhanced by increased temperature and moisture, and as a function of GV content in the mixture. The degree of methylation of the GV used is also an important factor, as grades with higher percentages of the tetra- or penta-methyl pararosaniline chloride forms of GV (hexa-methyl pararosaniline chloride) cause more rapid degradation during sterilization cycles.

Accordingly, it is desirable to provide an antimicrobial medical device capable of overcoming the disadvantages described herein at least to some extent.

### SUMMARY OF THE INVENTION

The foregoing needs are met, to a great extent, by the present invention, wherein in one respect an antimicrobial medical device and method of making an infection resistant medical article is provided.

An embodiment of the present invention pertains to a medical device. The medical device includes a first region having a biguanide or a pharmaceutically acceptable salt thereof and a second region having a Lewis acid.

Another embodiment of the present invention related to a method of making an infection resistant medical article. The method includes the steps of generating a first region having a biguanide or a pharmaceutically acceptable salt thereof and generating a second region having a Lewis acid.

Yet another embodiment of the present invention pertains to a medical device. The medical device including a first region, second region, and third region. The first region includes a chlorhexidine or a pharmaceutically acceptable salt thereof. The second region includes a gentian violet. The third region is disposed between the first region and the second region to separate the first region from the second region.

There has thus been outlined, rather broadly, certain embodiments of the invention in order that the detailed description thereof herein may be better understood, and in order that the present contribution to the art may be better appreciated. There are, of course, additional embodiments of the invention that will be described below and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1 is a high pressure liquid chromatograph showing an analysis of chlorhexidine and Gentian violet.

FIG. 2A is a high pressure liquid chromatograph showing an analysis of Gentian violet from a first supplier.

FIG. 2B is a high pressure liquid chromatograph showing an analysis of Gentian violet from a second supplier.

FIG. 2C is a high pressure liquid chromatograph showing an analysis of Gentian violet from a third supplier.

FIG. 3 is a chemical structure of several pararosanilin chlorides.

FIG. 4 is a high pressure liquid chromatograph showing an analysis of chlorhexidine extracted from humidified polyurethane incubated at 55° C. for 20 days.

FIG. 5 is a high pressure liquid chromatograph showing an analysis of Gentian violet extracted from humidified polyurethane incubated at 55° C. for 20 days.

FIG. 6 is a high pressure liquid chromatograph showing an analysis of 3.07% chlorhexidine and 1% Gentian violet incubated at 55° C. at 100% humidity.

FIG. 7 is a graph showing a mass spectrometry analysis of 3.07% chlorhexidine and 1% Gentian violet incubated at 55° C. at 100% humidity.

FIG. 8 is a graph showing amount of degradation product as a function of varying temperature and Gentian violet concentration in dry conditions.

FIG. 9 is a graph showing amount of chlorhexidine degradation product as a function of varying temperature and Gentian violet concentration in moist conditions.

FIG. 10 is a diagram showing a proposed degradation mechanism and degradation structure for mixtures of chlorhexidine and Gentian violet.

FIG. 11 is a graph showing amount of degradation product as a function of ethylene oxide sterilization and varying Gentian violet supplier.

FIG. 12 is a high pressure liquid chromatograph showing an analysis of chlorhexidine and Gentian violet extracted from a sample of three layer tubing according to an embodiment of the invention.

FIG. 13 is a high pressure liquid chromatograph showing an analysis of chlorhexidine and Gentian violet extracted from a sample of five layer tubing according to another embodiment of the invention.

FIG. 14 is a high pressure liquid chromatograph showing an analysis of chlorhexidine and Gentian violet extracted from a sample of humidified ethylene oxide sterilized three layer tubing according to an embodiment of the invention.

FIG. 15 is a graph showing an absence of degradation of Chlorhexidine from layered constructs prepared according to an embodiment of the invention after 20 days aging at 40° C. and 75% relative humidity.

### DETAILED DESCRIPTION

Embodiments of the invention provide infection resistant medical articles with two or more segregated antimicrobial agents and methods of segregating antimicrobial agents. To segregate the antimicrobial agents, the agents are incorporated into different layers or portions of the article. Generally, the antimicrobial agents include any suitable reagent having antimicrobial properties. It is an advantage of some embodiments that agents having negative interactions may be incorporated into the article while reducing the aforementioned negative interactions. In a particular embodiment, a biguanide may be segregated from a Lewis acid in a medical article. The biguanide may include any suitable agent such as chlorhexidine, alexidine, polyhexamethyl biguanide (PHMB), or the like or a pharmaceutically acceptable salt thereof. In addition, the biguanide may include a combination of two or more biguanides. The Lewis acid may include any suitable Lewis Acid agent. In general, suitable Lewis acids include methyl donors, triarylmethane dyes, and the like. A particular example of a suitable Lewis acid includes antimicrobial dyes such as methyl violet, brilliant green (BG), and the like. As disclosed herein, a particularly suitable methyl violet includes the hexamethylated form, Gentian violet (GV). However, it is to be understood that various other forms such as, tetramethylated, pentamethylated, and combinations thereof are suitable as well. Furthermore, the Lewis acid may include a combination of Lewis acids.

Embodiments of the present invention include an improvement enabling incorporation of various combinations of biguanides with Lewis acids as antimicrobial agents in shelf stable configurations into medical devices or articles by a segregating these agents into separate and distinct layers, regions or zones to preclude generation of degradation products. In another example, one or more biguanide and/or Lewis acid may be encapsulated in liposomes and subsequently suspended in a coating solution of another agent or mixed into a layer, region, or zone having another agent. In yet another example, one or more biguanide and/or Lewis acid may be independently liposomally incapsulated and the liposomes which may be combined in a coating, layer, region, or zone. In yet again another example, separate coatings, each having one of the agents, may be applied to the surface in a pattern which reduces or eliminates contact between the coatings such as, for example, non-overlapping stripes. More specifically, the coating may be applied via an inkjet type printing device. To further minimize contact between the coatings, a mask may be employed to reduce overspray.

We have determined that the formation of the degradant requires contact between the biguanide and the Lewis acid. The separation of the agents stabilizes the biguanide. Such configurations can be produced for example by a multi-layer co-extrusion process, injection molding, or encapsulation. Suitable devices that may be improved by embodiments of the invention include catheters, tubes, sutures, non-wovens, meshes, drains, shunts, stents, foams, etc. Other suitable articles include those improved by incorporation of a broad spectrum antimicrobial and/or antifungal activity. These devices may be packaged in a moisture resistant or moisture proof packaging such as, foil or Mylar® packaging to hinder inadvertent hydrolysis which initiates a cascade of degradation. In addition, the atmosphere within the packaging may be replaced with dry nitrogen, vacuum, or the like.

The benefit of the various embodiments of this invention is the incorporation of both biguanide and Lewis acid into a device without creating biguanide degradation products. Particular example is made of chlorhexidine degradation products resulting from interaction with GV in some of the following examples. However, further benefits of various embodiments of the invention extend to improving compatibility issues with any suitable bioactive agent. Examples of suitable bioactive agents includes antibiotics, other antiseptic agents, antithrombogenic agents, anticoagulants, fibrinolytics, anti-inflammatory agents, antifibrotic agents, antiproliferative agents, pain relief medications, chemotherapy agents, antibodies, peptide and peptide mimetics, nucleic acids, and the like.

The microbiologic benefit of directly or freely mixed chlorhexidine and GV has been previously determined. However, the stability of this mixture has not been studied. We have found that a degradation product can be produced when chlorhexidine is directly mixed with Gentian violet. The degradation product is undesirable in that it may reduce the potency of chlorhexidine, and have safety issues such as irritation, sensitization, allergenicity or toxicity. We have also found that the degradation is enhanced by increased temperature and moisture, and as a function of Gentian violet content in the mixture. The degree of methylation of the Gentian violet used is also an important factor, as grades with higher percentages of the tetra- or penta-methyl pararosaniline chloride forms of Gentian violet (hexa-methyl pararosaniline chloride) cause more rapid degradation during sterilization cycles.

In order to identify the degradation product, we analyzed a sample of freely mixed chlorhexidine and GV solution by liquid chromatography-tandem mass spectrometry (LC/MS/MS). It was determined that the molecular weight of the degradation product was 515 grams/mole. The likely structure of the degradation product, though not positively identified, has been elucidated based on this data. The hypothesized degradation pathway requires both moisture and a methyl donor (such as GV).

Degradation is reported in the following examples as measured by HPLC as peak area of the degradant peak divided by the total peak area of the chlorhexidine peak and the degradant peak (i.e. Peak Area_{degradant}/[Peak Area_{degradant}+Peak Area_{chlorhexidine}]*100). The peak areas are determined from integration of chromatograms.

### Methods and Results

### Example 1

A high pressure liquid chromatography (HPLC) analytical method was developed to quantify Gentian violet and chlorhexidine. Both compounds have ultraviolet or visible wavelength absorbance, and so were characterized using a UV-Vis diode array detector. Chlorhexidine and Gentian violet are identified by retention times versus a set of external standards. The external standards also form a calibration curve that is used to quantify the chlorhexidine and Gentian violet content in samples. The method is summarized as follows:

Instrument: Agilent 1200 Series DAD or Perkin Elmer Series 200 system
Column: Agilent Eclipse XDB-CN 5μ 4.6×150 mm column with an XDB-CN 5μ 4.6×12.5 mm guard column and an inline frit.
Program: A gradient program was run using two solvent reservoirs:

1. Mobile Phase A: 100% DI Water/0.2% Trifluoroacetic Acid

2. Mobile Phase B: 100% Acetonitrile/0.2% Trifluoroacetic Acid

### Solvent Gradient:

0-5 min 35% B

5-6 min 35-40% B

6-12 min 40% B

12-15 min 35% B

### Wavelength A=280 nm

### Wavelength B=588 nm

### LC Conditions:

Flow rate=1.0 ml/min

Pressure ˜1800 psi

Chlorhexidine analyzed at Wavelength A
Gentian Violet analyzed at Wavelength B

Standard Preparation:

Standards were made by preparing a stock solution of Chlorhexidine acetate (purchased from Sigma-Aldrich, St. Louise, Mo., USA referred as Sigma or Aldrich) and Gentian Violet (purchased from Sciencelabs, Kyle, Tex., USA, referred as Sciencelabs; Sigma-Aldrich referred to as Sigma, or Yantai Keyuan New Materials Co., Yantai, China, referred as Yantai) by weighing 0.50001 g of Gentian violet and 1.99999 g of Chlorhexidine acetate in a 100 mL Class A volumetric flask and diluting with 50% methanol. Serial dilutions were then made using 20% acetonitrile to obtain the following concentrations shown in Table 1:

TABLE 1
Chlorhexidine acetate/Gentian violet
external standard concentrations

| | GV | CHA |
|---|---|---|
| Std # | (μg/mL) | (μg/mL) |
| 1 | 1 | 4 |
| 2 | 10 | 40 |
| 3 | 50 | 200 |
| 4 | 100 | 400 |
| 5 | 200 | 800 |

A calibration curve was produced for Chlorhexidine acetate and each of the 2 or 3 Gentian violet peaks (according to purity). The calibration curves were constructed to correct for day to day variances in conditions such as pressure, and temperature variations.

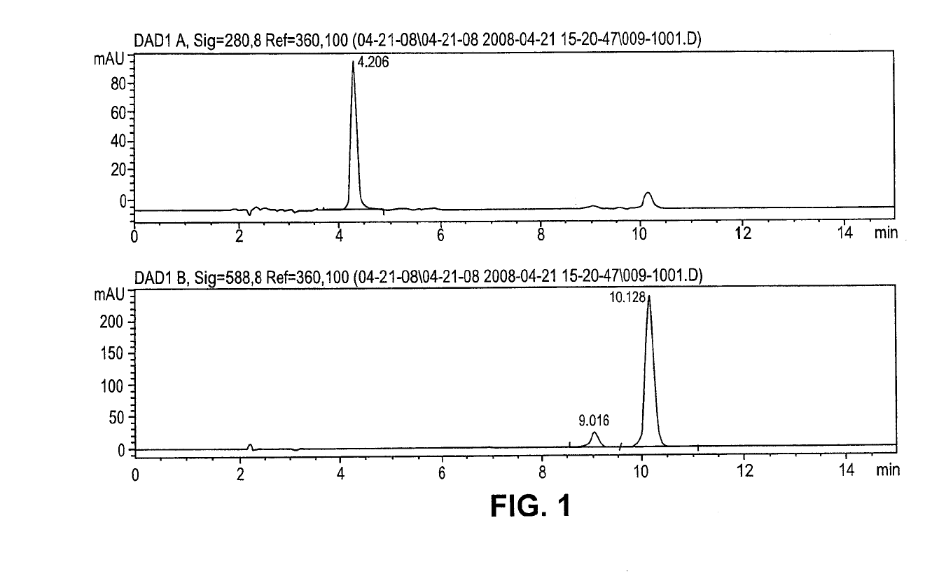

FIG. 1 is a typical chromatogram of chlorhexidine (upper graph) and Gentian violet (lower graph). The top chromatogram shows chlorhexidine at 280 nm and the bottom one shows Gentian violet at 588 nm. The Gentian violet shown was purchased from Yantai.

The sample preparation method, prior to HPLC analysis, is summarized as follows: samples were placed in centrifuge tubes, to which a volume of tetrahydrofuran (THF) was added (according to cited protocols or per examples). The tubes were vortexed until the sample was completely dissolved. For samples used as medical device coatings where a polymer was also present, a volume of deionized water was then added and the tubes were again vortexed for 10 minutes to precipitate the any polymer in the sample. The tubes were then centrifuged for 10 minutes at 4000 RPM to spin the polymer down out of the solution. A small amount of supernatant was transferred to an HPLC vial for analysis.

### Example 2

### Characterization of GV Purchased from Different Suppliers

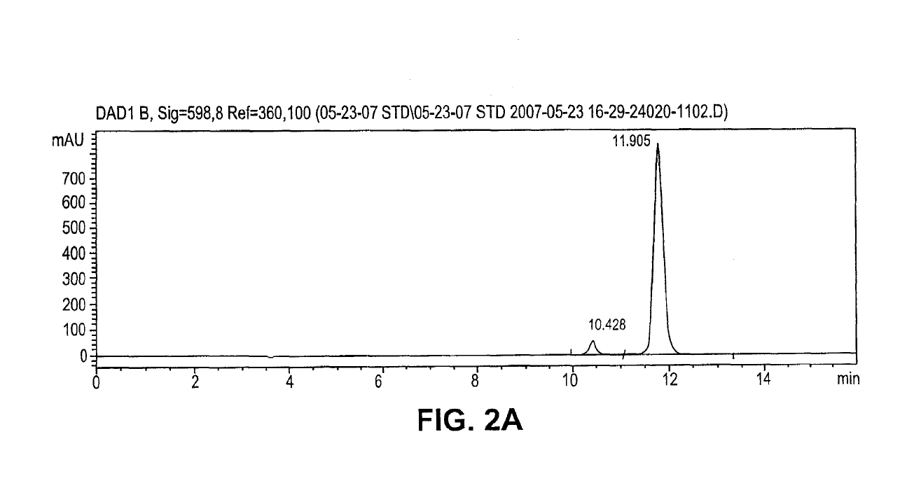

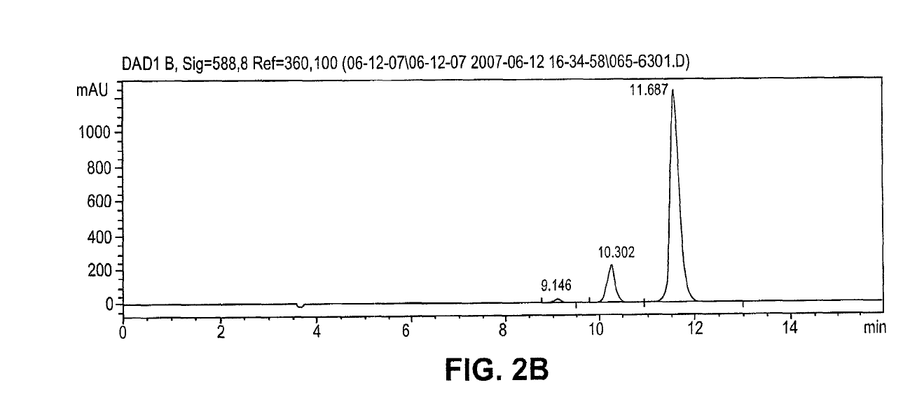

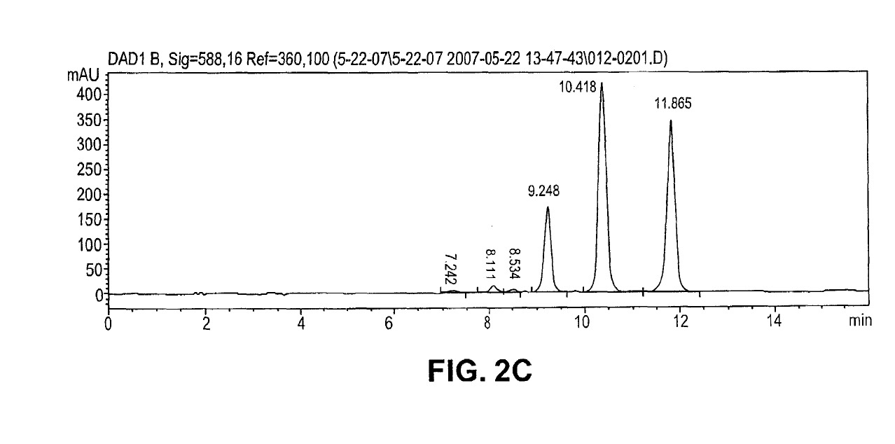

Purity of Gentian violet from different suppliers varies significantly. FIGS. 2A-2C are chromatograms showing HPLC analysis of Gentian violet from different suppliers. FIG. 2A is a chromatogram showing HPLC analysis of Gentian violet purchased from Yantai. FIG. 2B is a chromatogram showing HPLC analysis of Gentian violet purchased from Sigma/Aldrich. FIG. 2C is a chromatogram showing HPLC analysis of Gentian violet purchased from ScienceLab.

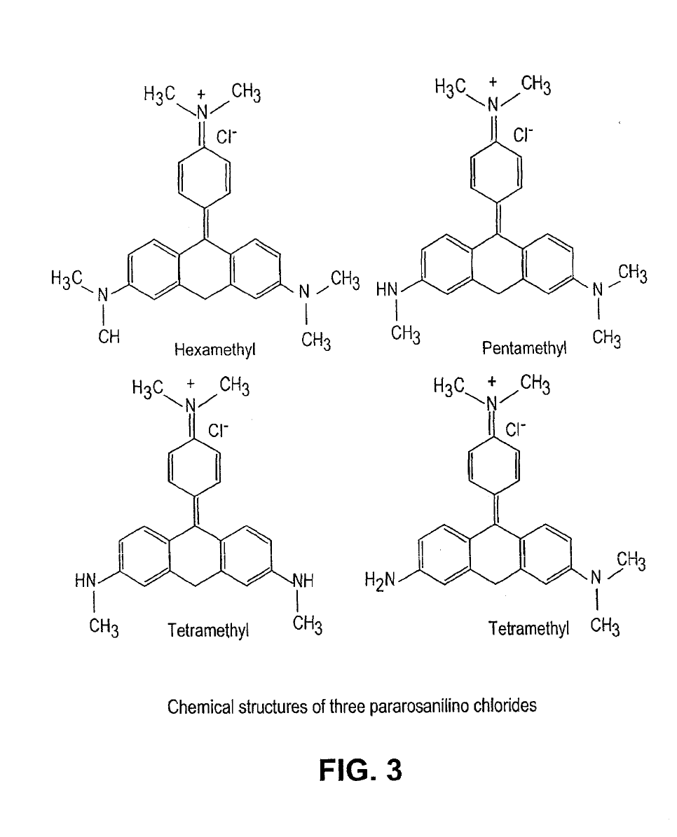

Based on MS (mass spectrometry) data, the molecular weights of the three major peaks were quantified. Chemical structures were attributed based on the molecular weights. They are hexamethylpararosaniline chloride, pentamethylpararosaniline chloride and tetramethylpararosaniline chloride (shown in FIG. 3). The retention time and molecular weight of these potential degradation products are summarized in Table 2.

TABLE 2
Molecular weight and retention time of pararosaniline chlorides

| | hexamethyl | pentamethyl | tetramethyl |
|---|---|---|---|
| Chemical formula | C₂₅H₃₀ClN₃ | C₂₄H₂₈ClN₃ | C₂₃H₂₆ClN₃ |
| Molecular weight | 407.5 | 393.5 | 379.5 |
| Retention Time | 11.9 | 10.4 | 9.2 |
| (min) | | | |

### Example 3

### Polyurethane Film Coating Procedure

Polyurethane films, used in the studies described in examples 4-6 and 9-11, were prepared as follows: 500 g of coating solution was prepared by dissolving 3.74% (w/w) Tecoflex 93A resin and 1.17% (w/w) Tecoflex 60D resin in a 74/26% mixture of THF/Methanol. The mixture was stirred until all of the resin was completely dissolved. 3.07% (w/w) chlorhexidine diacetate (CHA) was then added to the coating solution. Solutions containing different concentrations of GV were made by adding 0.1, 0.2, 0.4, 0.6, 0.8 or 1.0% (w/w) GV to 50 g aliquots of the CHA containing coating solution (or resin only solution in the case of GV only coating solution). 0.5 g of the CHA only, GV only, or 0.1-1.0% GV/CHA solutions was added to the bottom of a 30 mL glass vial and allowed to dry under ambient conditions overnight to evaporate the solvent and cast films in the vials. Vacuum dried samples were prepared by further drying the film containing vials at 25° C. under 30 in, Hg for 48 hours. Humidified samples were prepared by adding 75 μL of deionized water to the air dried samples and capping for the duration of the studies.

### Example 4

### CHA Stability in Polyurethane Films Containing no GV

Chlorhexidine diacetate (CHA) films were prepared according to example 2. Half of the vials were then spiked with 754, of deionized water and capped to create a 100% relative humidity environment, while the other half were vacuum dried (30 mm Hg) at 25° C. for 48 hours. The vials were then incubated in an oven at 25° C., 35° C., 45° C., and 55° C. for 20 days.

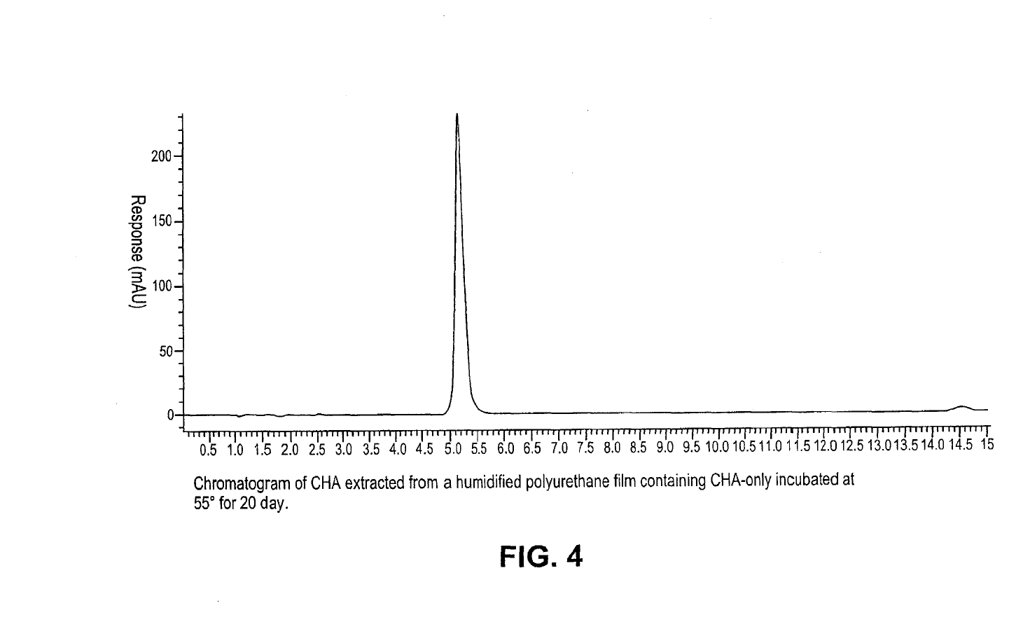

Samples were analyzed post air drying and post 20 day incubation. Sample analysis was performed using the method in Example 1. The vacuum dried and humidified samples containing CHA displayed a single peak and showed no degradation peaks in the chromatograms at any tested temperature after the 20 day incubation, as depicted in FIG. 4.

### Example 5

### GV Stability in Polyurethane Films Containing no CHA

Gentian violet only films were prepared according to example 2. Half of the vials were then spiked with 75 μL of deionized water and capped to create a 100% humidity environment, while the other half were vacuum dried (30 mm Hg) at 25° C. for 48 hours. The vials were then incubated in an oven at 25° C., 35° C., 45° C., and 55° C. for 20 days.

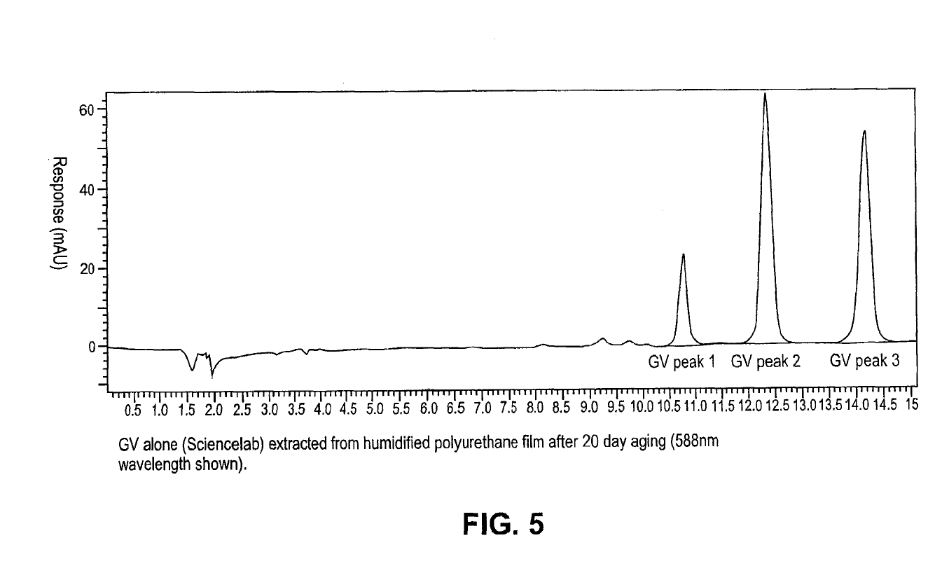

Samples were analyzed post air drying and post 20 day incubation. Sample analysis was performed using the method in Example 1. As illustrated in FIG. 5, the vacuum dried and humidified films containing only GV showed identical peaks in their chromatograms to the starting GV. Thus, no degradation products were detected in the chromatograms at any of the incubation temperatures after the 20 days.

Peak area ratios for pararosaniline chlorides are shown in Table 3 at different temperatures for 20 days. The peak area ratios for each temperature are calculated as follows: (Individual peak area)/Σ(individual peak)×100%. The variations are within the limits of experimental error further confirming the stability of GV in polyurethane films containing only GV.

TABLE 3
Peak area ratio for pararosaniline chlorides
under different temperature for 20 day

| Temperature | Tetramethyl | Pentamethyl | Hexamethyl |
|---|---|---|---|
| (° C.) | (%) | (%) | (%) |
| 0 day control | 14% | 44% | 42% |
| 25° C. at day 20 | 14% | 43% | 43% |
| 35° C. at day 20 | 13% | 43% | 44% |
| 45° C. at day 20 | 14% | 44% | 42% |
| 55° C. at day 20 | 12% | 43% | 45% |

### Example 6

### Formation of Degradation Peak in Polyurethane Films Containing Mixtures of CHA and GV

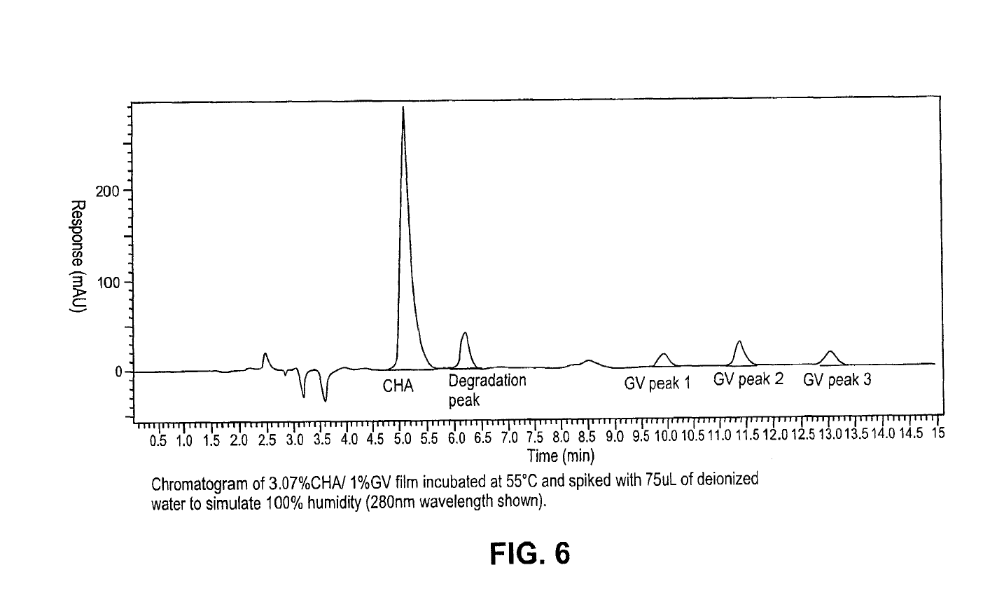

Polyurethane films loaded with both CHA and GV were prepared using the method in Example 3. Films containing a variety of GV to CHA ratios were prepared. Sample analysis was performed using the Method in Example 1. A degradation peak appears in polyurethane films containing both CHA and GV upon aging. FIG. 6 shows an example of a CHA/Sciencelab GV film illustrating the degradation peak.

### Example 7

### Identification of the Molecular Weight of the Degradant Peak

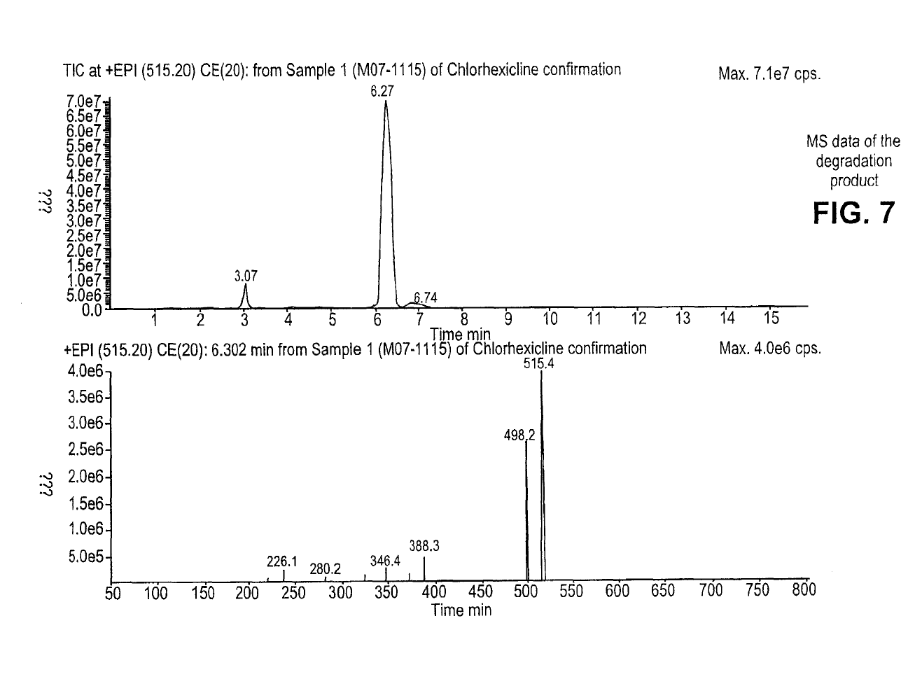

A sample of a CHA/GV solution containing the degradation product was analyzed by liquid chromatography and tandem mass spectroscopy (LC-MS/MS). This analysis determined that the molecular weight of the degradant peak was 515 g/mole. FIG. 7 shows the mass spectrometry (MS) data for the degradation peak.

### Example 8

### Effect of Temperature and GV Loading on CHA Degradation in Mixed CHA & GV Loaded Polyurethane Films

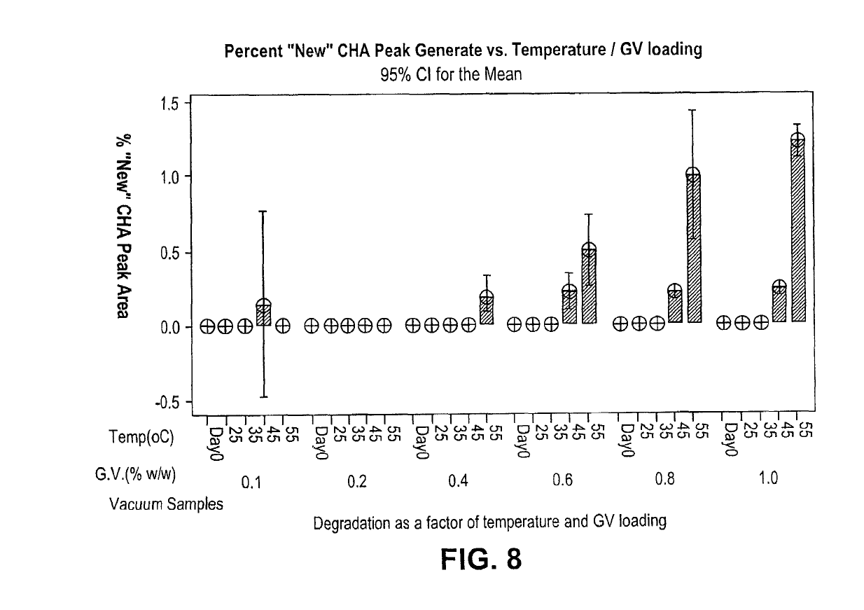

Chlorhexidine diacetate and Gentian violet containing films were prepared according to example 3. Samples were vacuum dried at 25° C. for 48 hours and then incubated under vacuum at 25° C., 35° C., 45° C., or 55° C. for 20 days. Samples were analyzed prior to and after incubation. FIG. 8 shows the results for analysis of the samples incubated for 20 days. Degradation begins at a GV loading of 0.4% (excluding the anomalous result at 0.1% GV and 45° C. which has a large uncertainty to a single outlier data point) and a temperature of 55° C. Degradation of CHA occurs at 45° C. upon increasing GV loading to 0.6 to 1.0%. The area of the degradation peak in the chromatogram also increases as GV loading increases. This data shows that not only is temperature a factor in causing degradation of CHA, but also that the amount of GV present is a factor as well.

### Example 9

### Effect of Moisture and GV Loading on Degradation of CHA in Mixed GV & CHA Containing Polyurethane Films

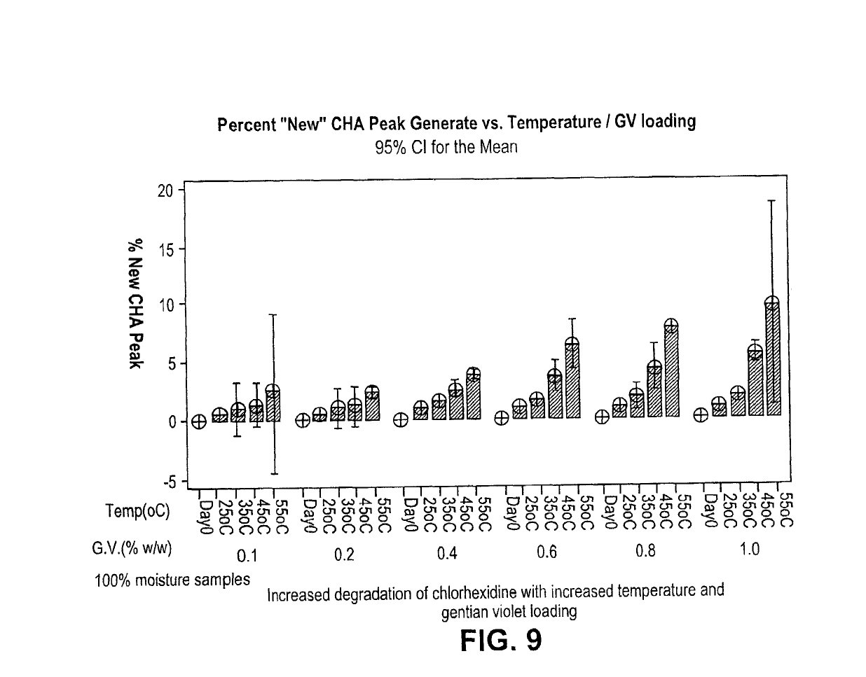

Chlorhexidine diacetate and Gentian violet containing films were prepared according to example 3. Samples were incubated in a 100% humidity environment at different temperatures for 20 days. Films were analyzed using the method on Example 1. Samples showed CHA degradation peaks that increased in magnitude with increasing temperature and with increasing percent GV in the cast films. The results from this experiment are shown in FIG. 9. The trend is very consistent with that shown in example 8; increasing GV content and increasing temperature are important factors in the degradation of CHA. Moreover, FIG. 9 shows that increased moisture very significantly accelerates the reaction between CHA and GV that creates the degradation product.

### Example 10

### Degradation Mechanism and Elucidation of the Degradation Product

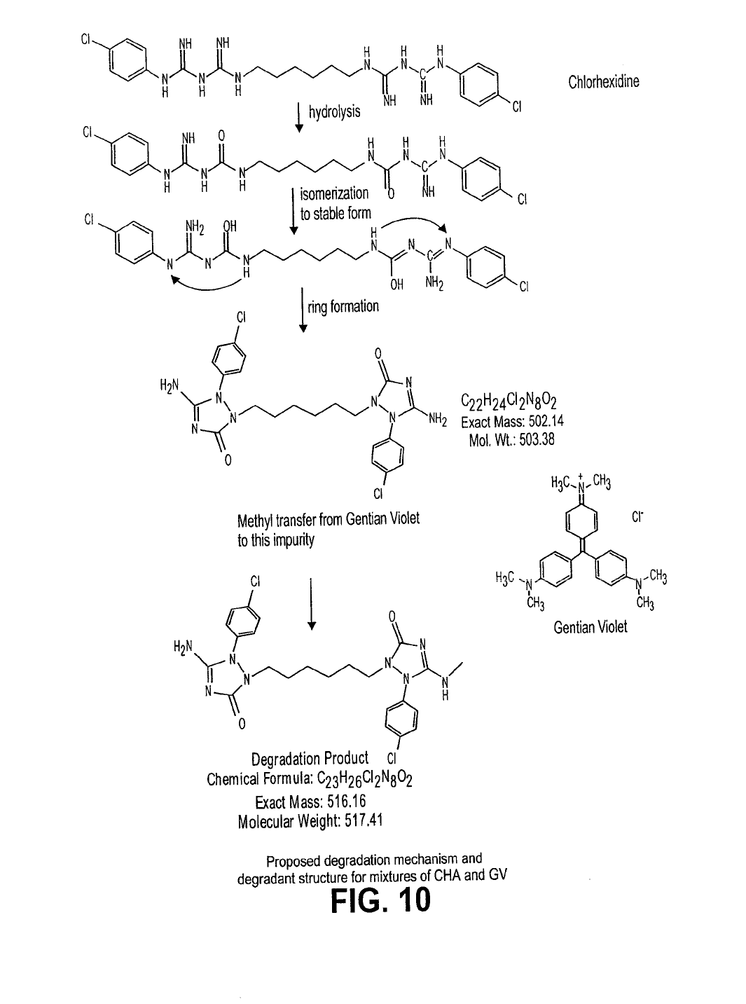

A proposed mechanism and structure of the degradation product based on the molecular weight data shown in example 7 as well as the results from examples 8 and 9 is shown in FIG. 10. As shown in FIG. 10, the degradation of chlorhexidine in the presence of Gentian violet is hypothesized to proceed through 4 steps illustrated sequentially in the diagram. The first is hydrolysis of one of the imine groups on chlorhexidine. This is followed by isomerization to form an unsaturated backbone. The third step is cyclization of the backbone to form a ring. The ring structure is then stabilized by methylation of the pendant amine. The source of the methyl group for this step is adjacent Gentian Violet

### Example 11

### CHA Stability in Polyurethane Films after Ethylene Oxide (ETO) Sterilization

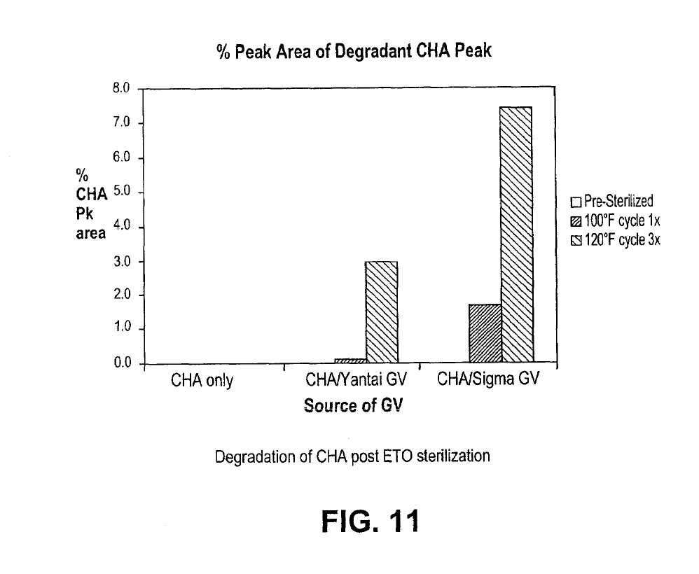

Samples were prepared using a bulk polymer coating solution of Tecoflex 93A/60D (3.74%/1.17% w/w) in 76% THF/24% MeOH to which 3.07% CHA (w/w) and 1% Gentian violet (Yantai or Aldrich) were added. 0.6 g of solution was added to glass vials and air dried, then vacuum dried at 25° C. for 48 hours. Vials were subjected to 3 ETO sterilization cycles at 120 F. The sterilization cycle involved at 60 minute humidification step followed by a 240 minute ETO exposure. Vials were also ETO sterilized a single cycle at 100° F. Extraction and analysis of the samples post sterilization was performed using the method of Example 1. FIG. 11 shows the results. Degradation was seen to increase with additional cycles, and also with the use of the Sigma GV, which has more tetra and penta-methyl pararosaniline chloride than the Yantai material. This data shows that polyurethane films containing only CHA is stable when sterilized, but not in combination with GV, and furthermore not in combination with less methylated (“less methylated” indicating a lower percentage of hexa-methyl pararosaniline chloride) GVs. FIG. 11 also shows that lowering the temperature and number of sterilization cycles decreases but does not prevent degradation of the chlorhexidine.

### Example 12

### Separation of CHA and GV in Different Layers by Co-extrusion

Three-layer extrusions of CHA and GV were produced by compounding 1% GV (Yantai) into Tecothane 95A resin on a Leistritz twin screw extruder. A sample of 8% CHA was compounded into low melt temperature Tecoflex resin on the same extruder. Compounded resins were then coextruded into 3-layer tubing constructs. The GV resin was gravity fed into a 1″ single screw extruder with barrel temperatures between 360-400° F. The CHA resin was starve fed into a 0.75″ single screw extruder with barrel temperatures between 260-275° F. The extrusions were drawn through a cold water bath and cut into lengths by an automatic cutter. The construct was extruded such that there was a thin layer of CHA containing polyurethane on either side of a thicker GV containing polyurethane core layer. This configuration limits the interaction of CHA and GV due to their separation into distinct layers.

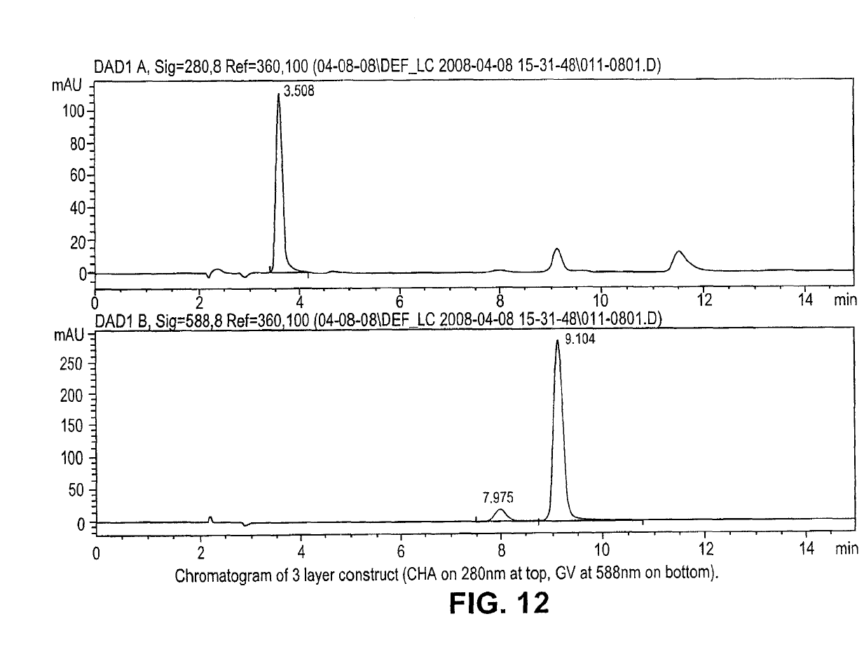

FIG. 12 shows a chromatogram of the three-layer construct after extrusion. Samples were prepared for analysis by dissolving the segment in 5.2 mL of THF by vortexing, and then adding 5.2 mL of deionized water. Samples were again vortexed to precipitate the polymer. The samples were then centrifuged prior to HPLC analysis. HPLC analysis was performed according to example 1. Unlike the single layer constructs where the GV and CHA were mixed, no degradation was detected from the co-extrusion of the construct (the peak at retention time of 11.5 minutes is from an additive in the THF solvent). Thus, as shown in FIG. 12, embodiments of the invention are capable reducing or eliminating chlorhexidine degradation products while providing the antimicrobial benefits of chlorhexidine and Gentian violet in a medical article. The peak at retention of 9.4 minutes on the top chromatogram is GV hexa-methyl species. Chlorhexidine diacetate is shown in the top chromatogram (280 nm) at retention time of 3.6 minutes. Gentian violet (Yantai) is shown in the bottom chromatogram (588 nm) at retention time of 8 minutes (penta-methyl species) and 9.4 minutes (hexa-methyl species).

### Example 13

### Separation of Compounds in Five Layers by Co-extrusion

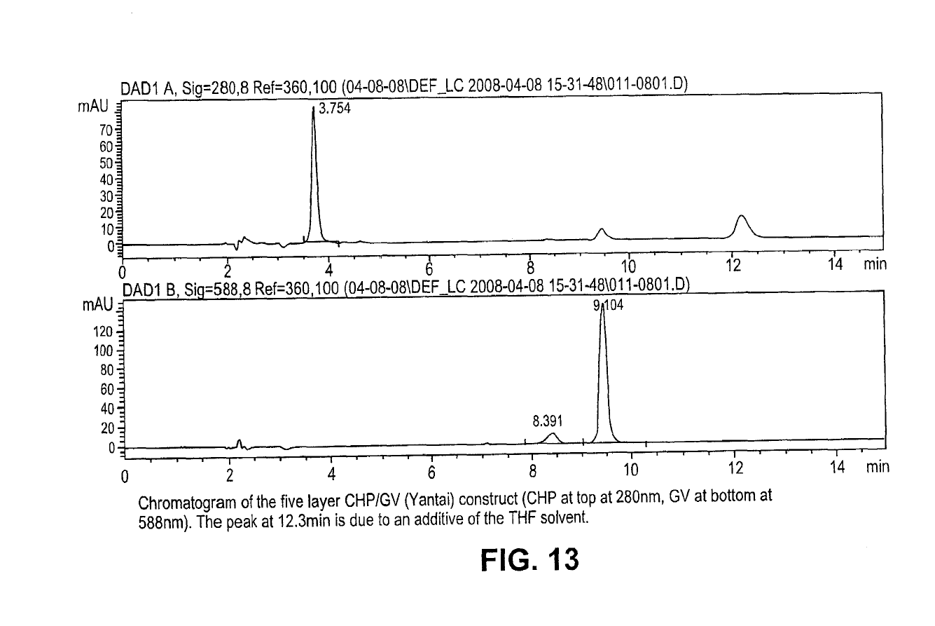

Samples were prepared in a similar manner as that described in example 12, but using 7% (w/w) compounded chlorhexidine palmitate (CHP) in place of CHA. This construct was configured so that there were 5 layers: an outer CHP containing layer, a drug free layer, a GV containing layer, another drug free layer, and an inner CHP containing layer. This configuration further separates chlorhexidine and/or various pharmaceutically acceptable salts thereof from GV. Extrusion samples were extracted with 10.4 mL THF and then 10.2 mL deionized water as per the method in example 1 and analyzed on the HPLC. A chromatogram of a sample of the extracted extrusion is shown in FIG. 13.

In various other embodiments, the co-extrusion may be performed as two or multiple layers and/or as longitudinal stripes or ‘candy cane-like’ along the medical device. Furthermore, it is envisioned that the co-extrusions may be axially disposed in and/or on the medical device.

### Example 14

### Stability of the Construct Post Sterilization

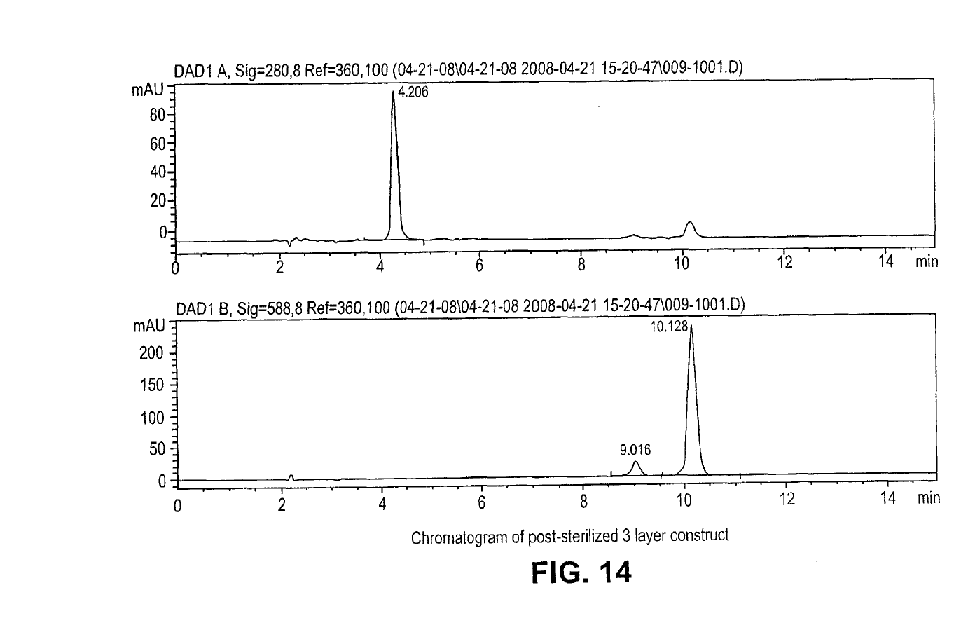

Three-layer extrusions were put into Tyvek® pouches and ETO sterilized. Vials were subjected to ETO sterilization at 120° F. The sterilization cycle involved a 60 minute humidification step followed by a 240 minute ETO exposure. FIG. 14 shows a chromatogram of the construct post sterilization. No degradation peak was detected, as in example 12.

### Example 15

### Stability of Construct after Aging

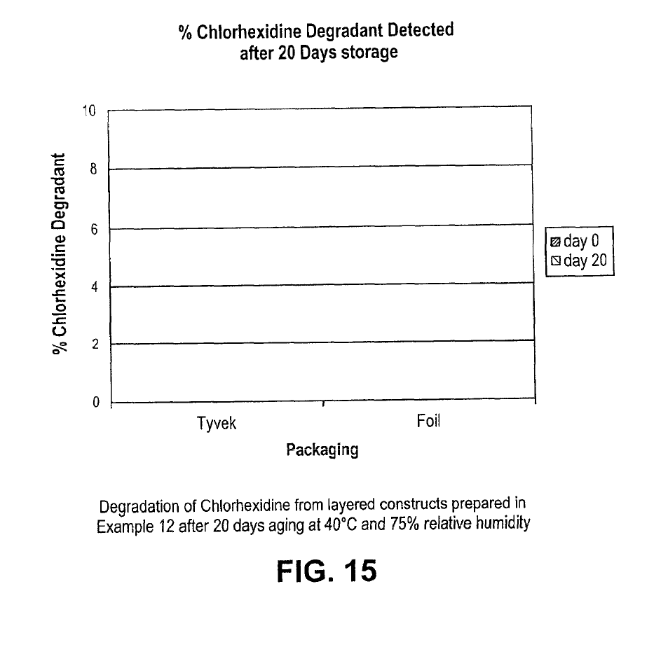

Three-layer construct samples prepared in Example 12 were packaged in Tyvek (breathable) or Foil (occlusive) pouches and placed into chambers at either 40° C./75% relative humidity for 20 days. Samples were analyzed after aging by extraction with 3 mL THF and 3 mL deionized water and analysis by HPLC according to example 1. FIG. 15 illustrates degradation of Chlorhexidine from layered constructs prepared in Example 12 after 20 days aging at 40° C. and 75% relative humidity. As shown in FIG. 15, no CHA degradation peak was detected in the 40° C./75% relative humidity aged samples in either Tyvek or foil pouches.

### Example 16

### Separation of Chlorhexidine and Gentian Violet Via Encapsulation

In another embodiment, encapsulation of CHA and GV may be utilized to separate CHA and GV in one or more layers. For example, microsphere samples of CHA and GV may be separately generated in any suitable manner. Of note, the term, ‘microsphere’ as used herein generally refers to any suitable sphere, spheroid, and/or particle from about 1000 μm to about 1 μm and smaller. As such, the term, ‘microsphere’ also generally refers structures known to those skilled in the art as, ‘nanosphere’ having a size of about 1000 ηm to about 1 ηm. These microsphere samples may be mixed prior to or during extrusion, or the samples may extruded separately.

Suitable methods of microsphere preparation may include spray drying, fluidized bed spray coating, spin coating, and the like. More particularly, suitable methods of microsphere preparation may include those employed by Harper International Corporation of Lancaster New York 14086-1698, U.S.A., Glatt International GmbH of Weimar Germany, and Angiotech Pharmaceuticals, Inc. of Vancouver Canada. In a specific example, microspheres may be prepared according to U.S. Pat. No. 6,224,794 entitled, Methods for Microsphere Production, the disclosure of which is incorporated herein in its entirety. In general the microsphere sample of CHA may include from about 5% CHA weight to volume (w/v) to about 50% w/v CHA or greater. In general the microsphere sample of GV may include from about 1% w/v GV to about 50% w/v GV or greater. In a preferred example, any suitable coating material utilized to produce the microsphere sample may have a melting temperature relatively higher than a base polymer to generate the medical device. Microsphere samples of CHA and GV may be compounded individually or together into a suitable low melt Tecoflex resin and extruded from a single screw extruder. In another example, the individual microsphere samples may be separately compounded into a base resin and co-extruded as described herein.

Our examples have shown that degradation products of chlorhexidine are formed when chlorhexidine or its salts and a methyl donor such as Gentian violet are directly combined and exposed to moisture. We have shown that by employing configurations that physically separate GV and CHA in the same device, medical devices can benefit from the combination of agents and avoid generating undesirable degradation products during processing, sterilization or storage. In the following examples, we have further determined that other biguanides react disadvantageously with other Lewis acids and would therefore benefit from segregation in accordance with embodiments of the invention.

### Example 17

### Brilliant Green-Chlorhexidine Formulation in THF/Methanol Solvent Mixture Cast Film

1.04 g Tecoflex 60D and 3.28 g Tecoflex 93A in 65 g were added in a glass jar containing 65 g of Tetrahydrofuran and stirred for four (4) hours at 45° C. Once all resin solids have dissolved, 23 g methanol added into the mixture and stirred for one (1) hour. 2.939 g chlorhexidine diacetate (CHA) was added into the solution mixture and stirred overnight at room temperature. Different Brilliant green (BG) concentrations (0.1 wt %-1.0 wt %) were added per the Table 4 below to an aliquot of the polymer-CHA solution and stirred until Brilliant Green dissolved:

TABLE 4
Brilliant green concentrations

| | Polymer solution | |
|---|---|---|
| Vial # | (g) | BG (g) |
| 1 | 10.108 | 0.01 |
| 2 | 5.5045 | 0.012 |
| 3 | 5.054 | 0.016 |
| 4 | 4.999 | 0.02 |
| 5 | 5.044 | 0.026 |
| 6 | 5.068 | 0.031 |
| 7 | 5.009 | 0.035 |
| 8 | 5.019 | 0.04 |
| 9 | 5.064 | 0.045 |
| 10 | 5.027 | 0.05 |

The green color of the solution mixture faded over time and chlorhexidine diacetate precipitated after 30 min of stirring at room temperature. Table 5 below lists different formulations of BG and CHA.

### Example 18

### Brilliant Green-Chlorhexidine formulation in DMF/THF Solvent Mixture

### Cast Film

In this experiment 40/60 THF/DMF solvent mixture was used instead of THF/Methanol from Example 17. Different Brilliant green (BG) concentrations (0.1 wt %-1.0 wt %) were added per the Table 5 below to an aliquot of the polymer-CHA solution (in Example 17) and stirred until Brilliant Green dissolved:

TABLE 5
Brilliant green concentrations

| | Polymer solution | |
|---|---|---|
| Vial # | (g) | BG (g) |
| 1 | 20.018 | 0.021 |
| 2 | 20.129 | 0.040 |
| 3 | 20.121 | 0.059 |
| 4 | 19.805 | 0.079 |
| 5 | 20.085 | 0.102 |
| 6 | 20.066 | 0.121 |
| 7 | 20.055 | 0.140 |
| 8 | 20.032 | 0.161 |
| 9 | 20.00 | 0.180 |
| 10 | 20.091 | 0.202 |

Similar observations were seen in DMF/THF solvent mixture as in experiment 17. The color solution mixture faded overtime and chlorhexidine diacetate precipitated after 30 min of stirring at room temperature.

### Example 19

### Brilliant Green, CHA, Gentian violet and Alexidine formulation by Spray coating

Polymer, Tecoflex 93A, resins were dissolved 70/30 DMF/THF solvent mixture at room temperature as in experiment 2. A pre-determined amount (listed in Table 6 below) of active agent (Alexidine, Brilliant Green, or Chlorhexidine diactate) was added in the polymer mixtures to make coating solutions.

TABLE 6
Coating formulation

| | Brilliant | | | | |
|---|---|---|---|---|---|
| | Green | CHA | Alexidine | TF-93A | DMF/THF |
| solution | (g) | (g) | (g) | (g) | 9 g) |
| 1 | | | 0.8 | 4 | 35.2 |
| 2 | | 1.6 | | 4 | 34.4 |
| 3 | 0.4 | | | 4 | 35.6 |

### Example 20

### Alexidine and Gentian Violet Constructs

A single layer 15 French Tecothane 95A extrusion containing Gentian Violet was prepared as described in Example 12. Multilayer coextrusions as in Example 12 were also prepared containing Gentian Violet in a core layer and polymer only top and bottom layers. The Gentian Violet extrusions were spray coated with the Alexidine composition (solution 1) of Example 19 and dried. Samples were stored at 100% relative humidity or 0% relative humidity for 20 days as described in Example 4 at a Temperature of 25 C. Alexidine Degradation was measured by the HPLC method of Example 1:

TABLE 7
Alexidine degradation

| | Starting | Alexidine content | |
|---|---|---|---|
| Storage | Alexidine content | after 20 days aging | % |
| condition | (μg/cm) | (μg/cm) | Degradation |
| Single layer - | 578 | 489.7 | 15.4% |
| 0% humidity | | | |
| Single layer - | 578 | 480.0 | 17% |
| 100% humidity | | | |
| Three layer - | 554 | 482.4 | 12.9% |
| 0% humidity | | | |
| Three layer - | 554 | 471.3 | 14.9% |
| 100% humidity | | | |

As shown in Table 7 above, more Alexidine degradation is seen in the single layer constructs where surface contact between Gentian violet and Alexidine is possible than in the 3 layer constructs.

### Example 21

### Brilliant Green+Chlorhexidine Coating

Tecothane extrusions were spray coated with Brilliant Green only (solution 3 Example 19) and with Brilliant Green and Chlorhexidine layers (solutions 2 and 3 Example 19). In the later sample, due to the time required for the solvents to evaporate, some extraction of chlorhexidine occurred into the Brilliant Green layer. Samples were stored for 20 days at 25 C under dry conditions (0% relative humidity). Analysis of Brilliant Green content was by HPLC (Example 1):

TABLE 8
Brilliant Green degradation

| | | Brilliant Green | |
|---|---|---|---|
| | Starting Brilliant | content after 20 | |
| | Green content | days aging | |
| Sample | (μg/cm) | (μg/cm) | % Degradation |
| Brilliant Green | 376.1 | 346.5 | 7.9% |
| only | | | |
| Brilliant Green + | 120.4 | 81.8 | 32.1% |
| Chlorhexidine | | | |
| layers | | | |

As shown in Table 8 above, more Brilliant Green degradation is seen in the construct where contact with Chlorhexidine is present.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

What is claimed is:

1. A medical device comprising:
a first region comprising a biguanide or a pharmaceutically acceptable salt thereof; and
a second region comprising a Lewis acid.

2. The medical device according to claim 1, wherein the biguanide is an Alexidine.

3. The medical device according to claim 1, wherein the biguanide is a chlorhexidine.

4. The medical device according to claim 1, wherein the biguanide is a polyhexamethyl biguanide (PHMB).

5. The medical device according to claim 1, wherein the biguanide includes a mixture of a plurality of biguanide agents.

6. The medical device according to claim 1, wherein the Lewis acid is a triarylmethane.

7. The medical device according to claim 6, wherein the triarylmethane includes brilliant green.

8. The medical device according to claim 6, wherein the Lewis acid includes a methyl violet.

9. The medical device according to claim 8, wherein the methyl violet is gentian violet.

10. The medical device according to claim 1, wherein the Lewis acid is a methyl donor.

11. The medical device according to claim 1, wherein the Lewis acid includes a mixture of a plurality of Lewis acids.

12. The medical device according to claim 1, wherein the first region is a first layer.

13. The medical device according to claim 12, wherein the second region is a second layer.

14. The medical device according to claim 13, wherein the first region and second region are co-extruded.

15. The medical device according to claim 14, further comprising:
a third layer disposed between the first layer and second layer.

16. The medical device according to claim 1, further comprising:
a multitude of first regions encapsulated in the medical device; and
a multitude of second regions encapsulated in the medical device.

17. The medical device according to claim 16, wherein the multitude of first regions and the multitude of second regions are microspheres.

18. The medical device according to claim 1, wherein the first region is a first stripe disposed longitudinally along the medical device.

19. The medical device according to claim 18, wherein the second region is a second stripe disposed longitudinally adjacent to the first stripe.

20. The medical device according to claim 1, wherein the medical device is a catheter, tube, suture, non-woven material, mesh, drain, shunt, or stent.

21. A method of making an infection resistant medical article, the method comprising steps of:
generating a first region comprising a biguanide or a pharmaceutically acceptable salt thereof; and
generating a second region comprising a Lewis acid.

22. The method according to claim 21, further comprising the step of:
selecting an Alexidine as the biguanide.

23. The method according to claim 21, further comprising the step of:
selecting a chlorhexidine as the biguanide.

24. The method according to claim 21, further comprising the step of:
selecting a polyhexamethyl biguanide (PHMB) as the biguanide.

25. The method according to claim 21, further comprising the step of:
selecting a mixture of a plurality of biguanides as the biguanide.

26. The method according to claim 21, further comprising the step of:
selecting a triarylmethane as the Lewis acid.

27. The method according to claim 26, further comprising the step of:
selecting a brilliant green as the triarylmethane.

28. The method according to claim 26, further comprising the step of:
selecting a methyl violet as the Lewis acid.

29. The method according to claim 28, further comprising the step of:
selecting a gentian violet as the methyl violet.

30. The method according to claim 21, further comprising the step of:
selecting a methyl donor as the Lewis acid.

31. The method according to claim 21, further comprising the step of:
selecting a mixture of a plurality of Lewis acids as the Lewis acid.

32. The method according to claim 21, further comprising the step of:
generating a first layer as the first region.

33. The method according to claim 32, further comprising the step of:
generating a second layer as the second region.

34. The method according to claim 33, further comprising the step of:
co-extruding the first region and second region.

35. The method according to claim 34, further comprising the step of:
co-extruding a third layer disposed between the first layer and second layer.

36. The method according to claim 21, further comprising the steps of:
encapsulating a multitude of first regions in the medical device; and
encapsulating a multitude of second regions in the medical device.

37. The method according to claim 36, further comprising the steps of:
generating a first multitude of microspheres including the biguanide; and
generating a second multitude of microspheres including the Lewis acid.

38. The method according to claim 35, further comprising the step of:
co-extruding a first stripe disposed longitudinally along the medical device.

39. The method according to claim 38, further comprising the step of:
co-extruding a second stripe disposed longitudinally adjacent to the first stripe.

40. The method according to claim 21, further comprising the step of:
fabricating a catheter, tube, suture, non-woven material, mesh, drain, shunt, or stent with the first region and second region.

41. A medical device comprising:
a first region comprising a chlorhexidine or a pharmaceutically acceptable salt thereof;
a second region comprising a gentian violet; and
a third region disposed between the first region and the second region to separate the first region from the second region.
